(19) United States
(12) Patent Application Publication
(10) Pub. No.: US 2025/0302337 A1
(43) Pub. Date: Oct. 2, 2025

(54) MEDICAL SUPPORT DEVICE, MEDICAL SUPPORT METHOD, AND MEDICAL SUPPORT PROGRAM

(71) Applicant: FUJIFILM CORPORATION, Tokyo (JP)

(72) Inventors: Ryohei KUWATSURU, Tokyo (JP); Yuki AMEMIYA, Tokyo (JP); Kohei ONODA, Tokyo (JP); Nobuya KITAMURA, Tokyo (JP)

(21) Appl. No.: 19/068,023

(22) Filed: Mar. 3, 2025

(30) Foreign Application Priority Data
Mar. 29, 2024 (JP) 2024-057943

(51) Int. Cl. A61B 5/11; A61B 5/00

(52) U.S. Cl. CPC A61B 5/1117; A61B 5/7275

(57) ABSTRACT

A processor acquires medical information including date information associated with a medical practice for a target patient and prediction date information for predicting a fall of the target patient, derives fall prediction information of the target patient using a prediction model that has been trained through machine learning to predict the fall of the target patient based on the number of elapsed days derived from the date information and the prediction date information, and notifies of the fall prediction information.

### CROSS REFERENCE TO RELATED APPLICATIONS

The present application claims priority from Japanese Patent Application No. 2024-057943, filed on Mar. 29, 2024, the entire disclosure of which is incorporated herein by reference.

### BACKGROUND

### Technical Field

The present disclosure relates to a medical support device, a medical support method, and a medical support program.

### Related Art

A fall is caused by muscle weakness due to aging or use of medications, and are a major incident that strongly affects a patient's quality of life (QOL) and life prognosis. Therefore, in order to prevent the patient from falling, various methods for predicting a fall have been proposed. For example, JP2006-228024A proposes a method for predicting a fall of a patient based on information acquired by sensors attached to the patient and shoes worn by the patient. In addition, JP2022-169193A proposes a method of predicting a fall using a learning model that has been trained to predict a fall based on patent's activity information, vital information, plan information such as a medical care plan, and record information including matters recorded in a medical record, and notifying a medical worker of the prediction.

In the method disclosed in JP2006-228024A, the patient needs to wear a wearable terminal or the like equipped with a sensor. In addition, medical institutions need to manage an operation and inventory of the wearable terminals. Therefore, the method disclosed in JP2006-228024A places a heavy burden on both the patient and the medical institutions. On the other hand, in the method disclosed in JP2022-169193A, although the fall can be predicted based on various types of information, the accuracy of the fall prediction is low because the number of elapsed days from prescription date and time of a drug or treatment date and time to a timing of predicting the fall is not considered.

### SUMMARY OF THE INVENTION

The present disclosure has been made in view of the above circumstances, and an object of the present disclosure is to predict a fall with high accuracy while reducing a burden on patients and medical institutions.

According to the present disclosure, there is provided a medical support device comprising: a processor, in which the processor acquires medical information including date information associated with a medical practice for a target patient and prediction date information for predicting a fall of the target patient, derives fall prediction information of the target patient using a prediction model that has been trained through machine learning to predict the fall of the target patient based on the number of elapsed days derived from the date information and the prediction date information, and notifies of the fall prediction information.

According to the present disclosure, there is provided a medical support method comprising: acquiring medical information including date information associated with a medical practice for a target patient and prediction date information for predicting a fall of the target patient; deriving fall prediction information of the target patient using a prediction model that has been trained through machine learning to predict the fall of the target patient based on the number of elapsed days derived from the date information and the prediction date information; and notifying of the fall prediction information.

According to the present disclosure, there is provided a medical support program causing a computer to execute: a procedure of acquiring medical information including date information associated with a medical practice for a target patient and prediction date information for predicting a fall of the target patient; a procedure of deriving fall prediction information of the target patient using a prediction model that has been trained through machine learning to predict the fall of the target patient based on the number of elapsed days derived from the date information and the prediction date information; and a procedure of notifying of the fall prediction information.

According to the present disclosure, it is possible to predict a fall with high accuracy while reducing a burden on patients and medical institutions.

### BRIEF DESCRIPTION OF THE DRAWINGS

FIG. 1 is a diagram showing a schematic configuration of a medical support system to which a medical support device according to the present embodiment is applied.

FIG. 2 is a diagram showing a hardware configuration of the medical support device according to the present embodiment.

FIG. 3 is a functional configuration diagram of the medical support device according to the present embodiment.

FIG. 4 is a diagram showing training data used for training a prediction model.

FIG. 5 is a diagram showing a list of high-risk patients.

FIG. 6 is a diagram showing a displayed contribution degree.

FIG. 7 is a diagram showing a message included in a notification.

FIG. 8 is a diagram showing a reservation patient list.

FIG. 9 is a flowchart showing processing performed in the present embodiment.

### DETAILED DESCRIPTION

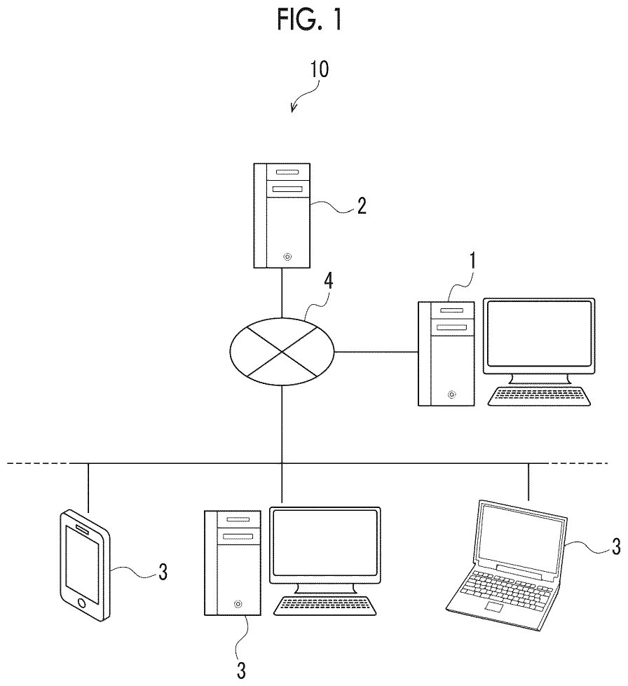

Hereinafter, an embodiment of the present disclosure will be described with reference to the drawings. First, a configuration of a medical support system to which a medical support device according to the present embodiment is applied will be described. FIG. 1 is a diagram showing a schematic configuration of the medical support system. In a medical support system 10 shown in FIG. 1, a medical support device 1, a management server 2, and a plurality of client terminals 3 are connected to each other via a network 4 in a communicable manner. The medical support system 10 is a system used for at least one facility (for example, a hospital) that handles a plurality of pieces of medical information.

The medical support device 1 predicts a fall of a patient as described below based on information acquired from the management server 2. A detailed configuration of the medical support device 1 will be described below.

The management server 2 includes a server computer or the like that manages various types of information relating to a plurality of patients. The management server 2 may be a cloud server. The management server 2 manages an electronic medical record of the patient, a medical image acquired by imaging the patient, a document created by a medical worker, and the like. The medical image is a medical image of the patient acquired by various modalities. Examples of the document include a report created by a doctor, a technician, a pharmacist, a nurse, or the like, and a report created by a modality that acquired the medical image of the patient.

The management server 2 acquires basic patient information, disease information, drug information, surgery information, and examination information from the electronic medical record, the medical image, and the document for the plurality of patients, and manages the acquired basic patient information, disease information, drug information, surgery information, and examination information as a database. In addition, in the present embodiment, the management server 2 acquires the keyword information by referring to the electronic medical record, and includes the keyword information in the medical care to manage the keyword information as a database.

The basic patient information includes information such as an age, a sex, a blood type, a height, a weight, BMI, medical departments with a history of a medical examination and the number of times of the medical examination, the number of visits to a hospital, a hospital visit time zone (average), the most recently visited medical departments, the number of times of hospitalization, the most recent hospitalization date, a hospitalization period, and the most recent discharge date.

The disease information includes the number of times a fall-related disease name is assigned, a disease name in units of ICD-10 Code or in a major classification units of ICD-10 Code (for example, M for musculoskeletal system), and a date on which the disease name is assigned (diagnosis date). In addition, the above-mentioned disease information is acquired for each of a definite disease name that is given by a doctor after a reliable diagnosis and a suspected disease name that is given in a state where a possibility of a disease is suspected in consideration of symptoms of the patient, examination results, and the like. The ICD-10 is the 10th revised edition of the international statistical classification of diseases and related health problems, which is a medical classification list of the World Health Organization.

The drug information includes the number of times a fall-related drug is prescribed (for example, “antianxiety drug” three times), a prescription date of the fall-related drug (for example, an antianxiety drug), the number of days in a case in which the drug is consecutively administered, the total number of drugs prescribed by a reference date, the total number of drugs and the total number of types of drugs prescribed up to the reference date, and the like. In addition, the drug information includes side effect information indicating the presence or absence of a side effect for each drug for each side effect. For the fall-related drug, a drug having the side effect information related to the fall (for example, “fainting”, “dizziness”, and “nausea”) may be derived based on the side effect information. An injection is treated in the same manner as the prescription of the drug. The prescription date may include a time in addition to the date.

The examination information includes whether a sample examination item is examined, an examination date of a sample examination item, the number of times a sample examination is performed for each medical department, the number of times a value exceeds or falls below an examination reference value, the total number or average of examination items, the total number or average of types of examination items, and the like. In addition to the sample examination, the same information is also included for results of a physiological examination, endoscopy, a radiological examination, and the like. The examination date may include a time in addition to the date.

The surgery information includes whether the patient has had a history of surgery (number of times), a surgery date, a surgery method, and the like. The surgery date may include a time in addition to the date.

The keyword information includes the number of medical records written up to a reference date, the presence or absence of a fall-related keyword (for example, “fall”, “collapse”, “dizziness”, “unsteadiness”, “cane”, and “walker”), the total number of times of appearance of the keywords, and the like.

The management server 2 acquires the medical information from the database and provides the medical information to the medical support device 1 via the network 4 in response to a request from the medical support device 1, which will be described below.

The client terminal 3 is a terminal device owned by a medical worker such as a doctor, a technician, a pharmacist, a nurse, and other hospital staff. Examples of the client terminal 3 include a workstation, a personal computer, a tablet terminal, a smartphone, and a smartwatch.

Examples of the network 4 include a wide area network (WAN). The WAN is merely an example, and the network 4 may be composed of at least one of a local area network (LAN), a WAN, or the like.

The medical support system 10 may have an electronic medical record server that manages an electronic medical record, an image management server that manages an image, and a document management server that manages a document, instead of the management server 2, and these servers may be connected to each other via the network 4 in a communicable manner.

In addition, in the example shown in FIG. 1, the client terminal 3 is connected to the medical support device 1 via the single network 4, but the technology of the present disclosure is not limited to this. For example, the client terminal 3 may be connected to the medical support device 1 via a network different from the network 4 to which the management server 2 is connected.

A medical support program according to the present embodiment is installed in the medical support device 1. The medical support device 1 may be a workstation or a personal computer installed in a hospital, or may be a server computer. The medical support program is stored in a storage device of another server computer connected to the network or a network storage (neither shown) in a state of being accessible from an outside, and is downloaded and installed in the medical support device 1 upon request. Alternatively, the medical support program is distributed by being recorded on a recording medium such as a digital versatile disc (DVD) or a compact disc read only memory (CD-ROM) and is installed in the medical support device 1 from the recording medium.

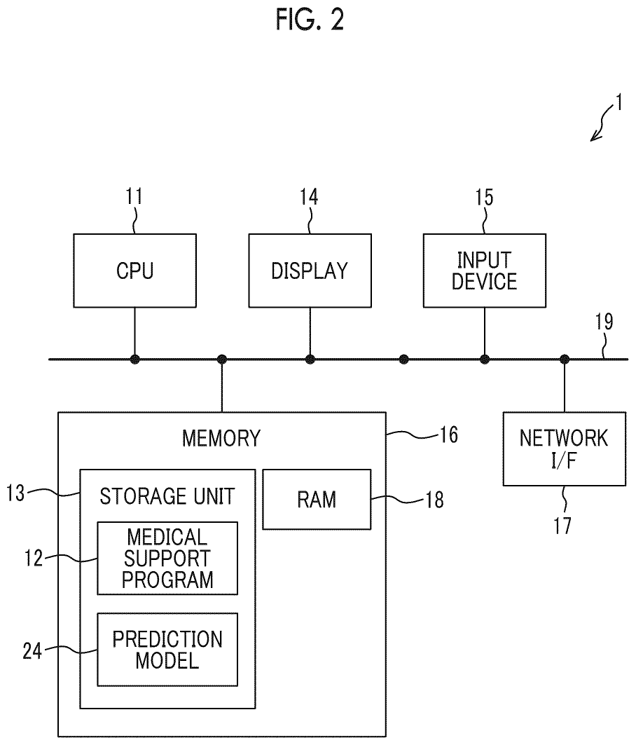

FIG. 2 is a diagram showing a hardware configuration of the medical support device according to the present embodiment. As shown in FIG. 2, the medical support device 1 includes a central processing unit (CPU) 11, a display 14, an input device 15, a memory 16, and a network interface (I/F) 17 connected to the network 4. The CPU 11, the display 14, the input device 15, the memory 16, and the network I/F 17 are connected to a bus 19. The CPU 11 is an example of a processor in the present disclosure.

The memory 16 includes the storage unit 13 and a random access memory (RAM) 18. The RAM 18 is a memory for primary storage and is, for example, a RAM such as a static random access memory (SRAM) or a dynamic random access memory (DRAM).

The storage unit 13 is a non-volatile memory, and is implemented by at least one of, for example, a hard disk drive (HDD), a solid state drive (SSD), an electrically erasable and programmable read only memory (EEPROM), or a flash memory. The storage unit 13 as a storage medium stores a medical support program 12 according to the present embodiment. The CPU 11 reads out the medical support program 12 from the storage unit 13, loads the medical support program 12 into the RAM 18, and executes the loaded medical support program 12.

The display 14 is a device that displays various screens and is, for example, a liquid crystal display or an electro luminescence (EL) display. The input device 15 is a device for a user to provide input and is, for example, at least any of a keyboard, a mouse, a microphone for voice input, a touchpad for proximity input including a contact, or a camera for gesture input. The network I/F 17 is an interface for connecting to the network 4.

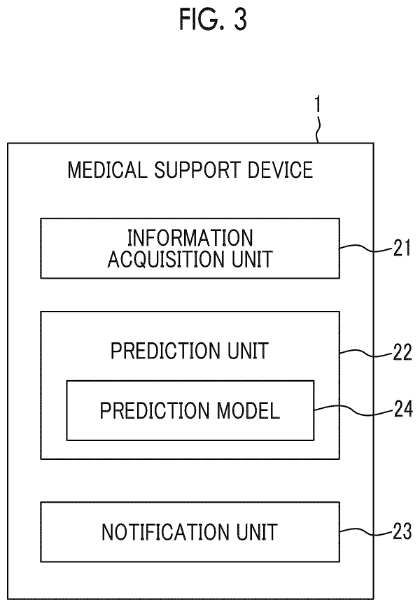

Next, a functional configuration of the medical support device according to the present embodiment will be described. FIG. 3 is a diagram showing the functional configuration of the medical support device according to the present embodiment. As shown in FIG. 3, the medical support device 1 comprises an information acquisition unit 21, a prediction unit 22, and a notification unit 23. In a case in which the CPU 11 executes the medical support program 12, the CPU 11 functions as the information acquisition unit 21, the prediction unit 22, and the notification unit 23.

The information acquisition unit 21 acquires, from the management server 2, medical information including date information associated with a medical practice for a target patient for which the fall is predicted and prediction date information for predicting the fall of the target patient, at a predetermined timing. Here, in the management server 2, the medical information for a plurality of patients is managed, but the information acquisition unit 21 acquires the medical information for a patient for whom the prediction date information is associated as a scheduled hospital visit date as a target patient. Therefore, the number of the target patients is smaller than the number of patients managed in the management server 2.

The prediction date information is information indicating a date on which a fall is predicted for the target patient, as described below, and examples thereof include a scheduled hospital visit date on which the target patient is scheduled to visit a hospital next time. The prediction date information may include information on a time such as a reservation time for a visit to a hospital in addition to the date. In addition, the prediction date information is not limited to a single date and includes a case of a plurality of dates. In the case of a plurality of dates, for example, the dates may be consecutive dates designated by a start date and an end date, or non-consecutive dates such as every Tuesday of the following month.

In the present embodiment, the information acquisition unit 21 acquires the medical information for all the target patients who have a reservation to visit a hospital on the day, that is, for whom the prediction date information is the day, for example, at a timing before a reception time on the day (for example, the night before). The information acquisition unit 21 may acquire, at a timing at which the target patient makes a reservation for the next hospital visit, the medical information and the prediction date information for the target patient.

Examples of the medical practice for the patient include at least one of prescription of a drug, an examination, surgery, hospitalization and discharge, or a diagnosis. The scope of the medical practice may include a medical practice that affects a fall. Determination of whether or not the medical practice affects a fall may be made based on the presence or absence of a specific description such as dizziness in the description of the side effect in the medical record, for example, and may be designated by the medical worker. In the present embodiment, the date information associated with the medical practice is at least one of a prescription date of a drug included in the drug information, an examination date of an examination included in the examination information of the medical information, a surgery date included in the surgery information, a hospitalization date and a discharge date of a patient included in the basic patient information, or a diagnosis date included in the disease information in the medical information.

In a case in which a prescription date for each of a plurality of types of drugs is included in the drug information, in a case in which an examination date for each of a plurality of types of examinations is included in the examination information, in a case in which a plurality of types of surgery dates are included in the surgery information, and in a case in which a plurality of hospitalization dates, a plurality of discharge dates, and a plurality of diagnosis dates are included in the basic patient information, in the present embodiment, at least one of the most recent prescription date, the most recent examination date, the most recent surgery date, the most recent hospitalization date, the most recent discharge date, or the most recent diagnosis date is used as the date information for the prediction date information. The prescription date of a drug may include a prescription start date and a prescription end date, and, in this case, the prescription end date need only be used as the date information. In a case in which the prescription date and the examination date include a time, the date information also includes information on the time.

In a case in which the medical information of the target patient includes all of the prescription date of the drug, the examination date, the surgery date, the hospitalization date, the discharge date, and the diagnosis date, a date closest to the prediction date information among these dates need only be used as the date information. In addition, one or a plurality of pieces of date information that are predetermined among the prescription date of the drug, the examination date, the surgery date, the hospitalization date, the discharge date, and the diagnosis date may be used. The most recent prescription date, the most recent examination date, the most recent surgery date, the most recent hospitalization date, the most recent discharge date, and the most recent diagnosis date are examples of specific date information of the present disclosure.

The prediction unit 22 derives fall prediction information of the target patient using a prediction model 24 that has been trained through machine learning to predict the fall of the target patient based on the number of elapsed days based on the date information and the prediction date information. The prediction model 24 is stored in the storage unit 13. In the present embodiment, the prediction unit 22 derives the number of elapsed days from a date based on the date information to a prediction date based on the prediction date information, from the date information and the prediction date information. For example, the number of elapsed days from at least one of the prescription date included in the drug information, the examination date included in the examination information, or the surgery date included in the surgery information to the prediction date based on the prediction date information is derived. In a case in which a plurality of pieces of date information among the prescription date, the examination date, and the surgery date are used, the prediction unit 22 derives a plurality of the number of elapsed days.

The prediction unit 22 may compare the number of elapsed days with a predetermined threshold value Th1 and derive the fall prediction information only in a case in which the number of elapsed days is equal to or smaller than the threshold value Th1. The threshold value Th1 can be set to, for example, 10 days, but the present disclosure is not limited to this. A different threshold value Th1 may be used depending on the classification of the medical information associated with the date information. That is, a different threshold value Th1 may be used depending on whether the date based on the date information is the prescription date included in the drug information, the examination date included in the examination information, the surgery date included in the surgery information, the hospitalization date and the discharge date included in the basic patient information, or the diagnosis date included in the disease information. In addition, in a case in which there are a plurality of drugs, a different threshold value Th1 may be used for each prescription date of the drug. The threshold value Th1 is an example of a first threshold value of the present disclosure.

The medical information and the number of elapsed days are input to the prediction model 24. In a case in which multiple numbers of days elapsed are derived, a plurality of the number of elapsed days are input. Specifically, a feature amount vector representing the medical information and the number of elapsed days is input. The feature amount vector is a vector having, as elements, a plurality of pieces of information included in the medical information and the number of elapsed days. Then, the prediction model 24 outputs fall prediction information based on the input medical information and the input number of days elapsed. The prediction model 24 derives the fall prediction information based on the medical information and the number of elapsed days.

For this reason, the prediction model 24 is constructed by training, through machine learning, a learning model using the medical information, the number of elapsed days from the day on which the medical practice is performed, and the information on the presence or absence of the fall as training data for a patient who has actually fallen. In the present embodiment, the training data is acquired from the database managed by the management server 2.

As the learning model for constructing the prediction model 24, for example, a decision tree model can be used. As the decision tree model, a light gradient boosting machine (LightGBM) model based on a gradient boosting algorithm may be used. LightGBM is a free and open-source distributed gradient boosting framework for machine learning. LightGBM is based on a decision tree algorithm, and is used for ranking, classification, and other machine learning tasks.

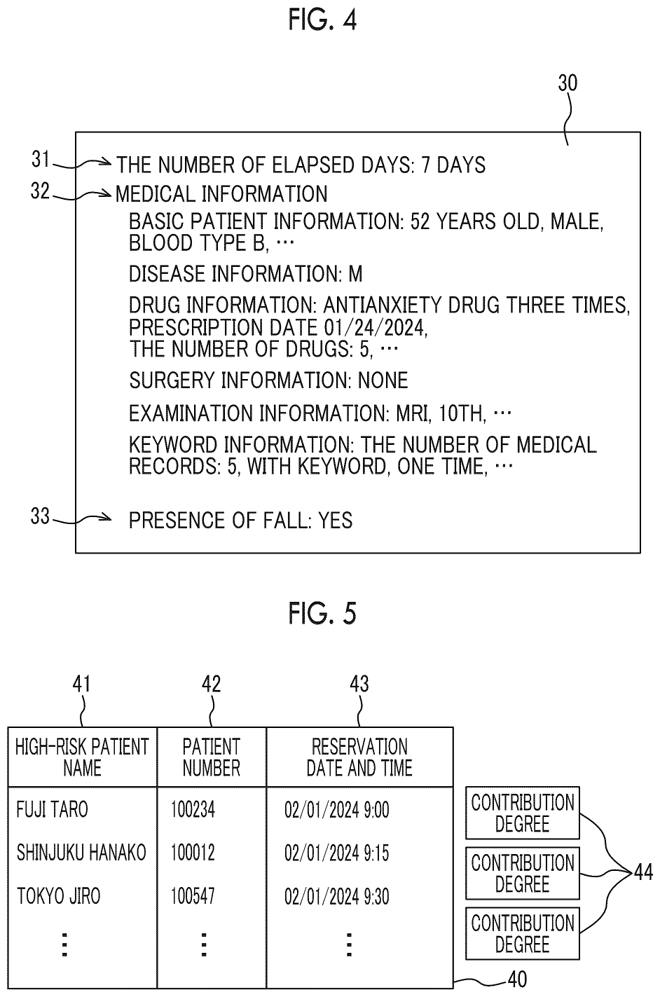

FIG. 4 is a diagram showing training data used for training the prediction model 24. As shown in FIG. 4, training data 30 includes the number of elapsed days 31 from the day on which the medical practice is performed, medical information 32, and information 33 on the presence or absence of the fall. In the training data 30 shown in FIG. 4, the number of elapsed days 31 is the number of elapsed days from the prescription date included in the drug information, and is, for example, 7 days. The medical information 32 is acquired for an actual patient used for the training data 30, and includes specific information of the basic patient information, the disease information, the drug information, the surgery information, the examination information, and the keyword information. The information 33 on the presence or absence of the fall is “Yes”. Multiple numbers of days elapsed days may be derived, in which case the number of elapsed days 31 of the training data 30 also includes multiple days.

In learning, the feature amount vector representing the number of elapsed days 31 and the medical information 32 is derived from the training data 30, and the derived feature amount vector is input to the learning model. The feature amount vector has dimensions corresponding to the number of elapsed days 31 and the number of pieces of the medical information 32. Then, the learning model is caused to output prediction probability of the fall as a value of 0 or more and 1 or less, for example. Then, the information 33 on the presence or absence of the fall included in the training data 30 is compared with the output value, and a difference therebetween is derived as a loss.

Correct answer data is 1 in a case in which there is a fall and is 0 in a case in which there is no fall. Then, the prediction model 24 is constructed by repeating the learning until the loss reaches a predetermined threshold value or less or until a predetermined number of times of learning is completed. The prediction model 24 constructed in this way outputs the prediction probability of the fall as a value of 0 or more and 1 or less in a case in which the number of elapsed days and the medical information are input. In general, the shorter the number of elapsed days from the medical practice, the higher the possibility of the fall. Therefore, as the number of elapsed days input to the prediction model 24 is smaller, the prediction probability of the fall output by the prediction model 24 is higher. On the other hand, the prediction model 24 may be constructed to output the presence or absence of the fall as a value of 1 or 0.

In the present embodiment, in a case in which the prediction unit 22 predicts the presence or absence of the fall, a method of SHapley Additive explanations (SHAP), which is a type of explainable AI technology, may be applied to the prediction model 24 to derive a contribution degree of each element (that is, a feature amount) of the feature amount vector input to the prediction model 24. SHAP is a method for obtaining contribution of each variable (feature amount) to a prediction result of a model, and is based on a concept called a Shapley value. The Shapley value was originally proposed in a field called cooperative game theory. In cooperative game theory, a main task is to determine how to fairly distribute rewards according to a contribution degree of each player in a game in which a plurality of players cooperate to clear the game to obtain rewards. In machine learning, since a prediction value is calculated by combining a plurality of types of feature amounts, the contribution degree of the feature amount to the prediction value can be derived by replacing the feature amount with the player and the rewards with the prediction value.

The notification unit 23 notifies of the fall prediction information derived by the prediction unit 22. The notification is made to the client terminal 3 of a predetermined medical worker, target patient, or family of the target patient via the network 4. For example, in a case in which the fall prediction information is derived before a reception time on the day, the notification unit 23 specifies the target patient (hereinafter, a high-risk patient) for whom the prediction information indicating a high possibility of the fall or the presence of the fall has been acquired. The high-risk patient is a target patient for whom the prediction probability of the fall output by the prediction model 24 is equal to or greater than a threshold value Th2, or a target patient for whom the prediction model 24 outputs the prediction result of the presence of the fall. The threshold value Th2 is an example of a second threshold value of the present disclosure.

In addition, the notification unit 23 may select the target patient in descending order of the fall prediction probability such that the number of the target patients is equal to or smaller than a threshold value Th3, instead of or in addition to the target patient for whom the prediction probability of the fall is equal to or greater than the threshold value Th2. The second threshold value Th2 and the third threshold value Th3 may be a predetermined set value or a variable value that varies according to input of the user or the like. For example, at least one of the second threshold value Th2 or the third threshold value Th3 may be set for each facility or each medical department.

The notification unit 23 creates a list of the specified high-risk patients and transmits the created list to the client terminal 3 of the medical worker associated with the target patient to notify of the fall prediction information. Examples of the medical worker associated with the target patient include a doctor in charge of the target patient, an outpatient nurse, a reception staff, and a hospital entrance staff.

FIG. 5 is a diagram showing a list of the high-risk patients. As shown in FIG. 5, a list 40 of the high-risk patients includes a high-risk patient name 41, a patient number 42, and a reservation date and time 43. The medical worker can refer to the list 40 and take measures to prevent the patient from falling down, such as stationing a staff at an entrance of the hospital to wait for the high-risk patient to come to the hospital.

In a case in which the contribution degree is derived by the prediction model 24, the list 40 includes a contribution degree button 44. In a case in which the contribution degree button 44 is selected, the contribution degree of the feature amount input to the prediction model 24, that is, the medical information and the number of elapsed days of the target patient to the fall prediction information is displayed on the client terminal 3. Instead of the display on the contribution degree button 44, the contribution degree may be displayed in advance in the list 40.

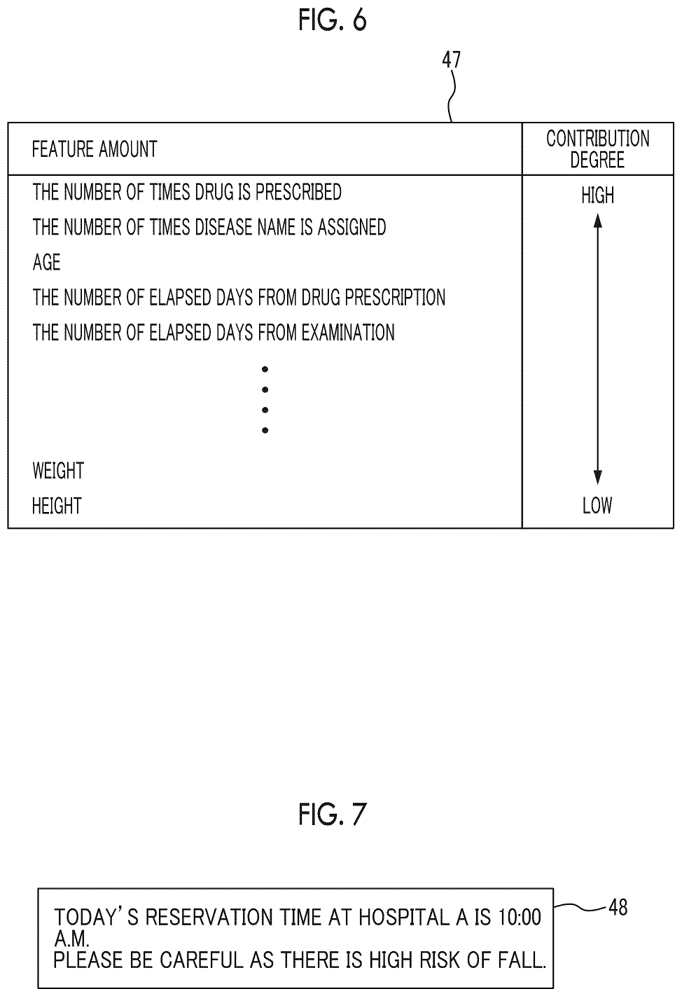

FIG. 6 is a diagram showing the displayed contribution degree. As shown in FIG. 6, in a contribution degree 47, the number of times the drug is prescribed, the number of times the disease name is assigned, the age, the number of elapsed days from the drug prescription, the number of elapsed days from the examination, the weight, and the height are displayed in descending order of the contribution degree. As described above, by notifying of the contribution degree, it is possible to know which feature amount of the medical information or the number of elapsed days has contributed to the fall prediction information.

In addition, the notification unit 23 may notify a terminal device of the high-risk patient himself/herself or the family that there is a high risk of the fall. FIG. 7 is a diagram showing an example of the notification. In FIG. 7, a notification 48 includes a message, “Today's reservation time at hospital A is 10:00 a.m. Please be careful as there is a high risk of fall”. The notification may be made by e-mail, a messaging application, a telephone, or the like. As a result, the target patient or the family of the target patient can pay more attention to the movement of the target patient and take measures to prevent the target patient from falling.

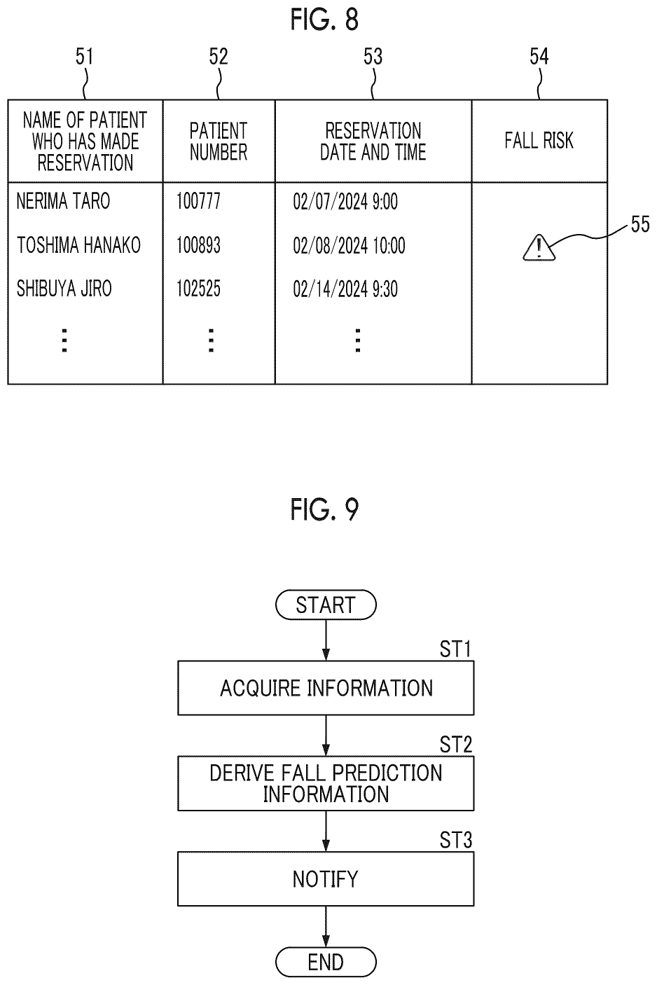

In addition, in a case in which the fall prediction information is acquired at a timing at which the target patient makes a reservation for the next hospital visit, a notification is made to the medical worker related to the target patient. The notification is made by adding an alert to the reservation patient list displayed on the client terminal 3 of the medical worker. FIG. 8 is a diagram showing another example of the notification. As shown in FIG. 8, a reservation patient list 50 includes a name of a patient 51 who has made a reservation to visit the hospital, a patient number 52, a reservation date and time 53, and a fall risk 54. In FIG. 8, for the high-risk patient, a mark 55 indicating a high fall risk is added to the fall risk 54. As a result, the medical worker can easily recognize which patient has a high fall risk among the patients who have made a reservation.

In the fall prediction information derived by the prediction unit 22, in a case in which there is no high-risk patient, the notification unit 23 may notify that there is no high-risk patient.

Next, processing performed in the present embodiment will be described. FIG. 9 is a flowchart showing processing performed in the present embodiment. The processing is started at a predetermined timing, and the information acquisition unit 21 acquires the medical information including the date information associated with the medical practice for the patient and the prediction date information for predicting the fall of the patient from the management server 2 (information acquisition; step ST1). Next, the prediction unit 22 derives the fall prediction information of the patient using the prediction model 24 that has been trained through machine learning to predict the fall of the patient (step ST2). Then, the notification unit 23 notifies of the fall prediction information (step ST3), and the processing ends.

As described above, in the present embodiment, the fall prediction information of the patient is derived based on the medical information including the date information associated with the medical practice and the prediction date information. Therefore, the patient does not need to wear a wearable terminal or the like equipped with a sensor, and the doctor does not need to manage the operation and inventory of the wearable terminals. In addition, it is possible to predict a fall taking into account the number of elapsed days from the medical practice to the timing of predicting the fall. Therefore, according to the present embodiment, it is possible to predict a fall with high accuracy while reducing a burden on patients and medical institutions.

Incidentally, it is known that the possibility of the fall increases in a case in which a fall-related drug is consecutively administered to a patient or in a case in which a fall-related drug is administered to a patient multiple times. For example, since a risk of osteoporosis is increased by long-term administration of a steroid agent, it is necessary to pay attention to the possibility of the fall. Therefore, the fall prediction information weighted according to the number of consecutive days of the medical practice, such as the number of consecutive days of administration of the drug, or the number of times of the medical practice, such as the number of times of administration of the drug within a predetermined period from the date represented by the reservation date information, may be derived.

In this case, the prediction unit 22 may perform weighting on the prediction probability of the fall output by the prediction model 24, or may perform weighting on the number of elapsed days input to the prediction model 24. In a case of performing the weighting on the prediction probability output by the prediction model 24, the fall prediction information may be derived by performing the weighting such that the prediction probability is larger as the number of consecutive days or the number of times of the medical practice is larger. Specifically, for the number of consecutive days, the fall prediction information need only be derived by multiplying the prediction probability by weight coefficients of 3.0, 2.0, and 1.0 for 30 days, 20 days, and 10 days, respectively. For example, in a case in which the number of consecutive days of administration of the fall-related drug is 30 days, in a case in which the prediction probability output by the prediction model 24 is 0.125, the prediction unit 22 need only derive the fall prediction information by performing an operation of 0.125×3.0=0.375. For the number of times of the medical practice, for example, the fall prediction information need only be derived by multiplying the prediction probability by weight coefficients of 3.0, 2.0, and 1.0 for 10 times, 5 times, and 3 times, respectively.

In addition, in a case of performing the weighting on the number of elapsed days with respect to the prediction model 24, the fall prediction information need only be derived by performing the weighting on the number of elapsed days such that the number of elapsed days input to the prediction model 24 is smaller as the number of consecutive days or the number of times of the medical practice is larger. Specifically, for the number of consecutive days, the fall prediction information need only be derived by multiplying the number of elapsed days by weight coefficients of 0.7, 0.8, and 0.9 for 30 days, 20 days, and 10 days, respectively. For the number of times of the medical practice, for example, the fall prediction information need only be derived by multiplying the prediction probability by weight coefficients of 0.7, 0.8, and 0.9 for 10 times, 5 times, and 3 times, respectively.

Here, the prediction probability output by the prediction model 24 decreases as the number of elapsed days decreases. Therefore, by performing the weighting on the number of elapsed days such that the number of elapsed days input to the prediction model 24 is smaller as the number of consecutive days or the number of times of the medical practice is larger, it is possible to derive the fall prediction information corresponding to the risk of the fall, such as the consecutive administration of the drug or the multiple times of administration.

On the other hand, the prediction model 24 may be constructed such that the prediction probability of the fall is derived in a case in which the number of consecutive days or the number of times of the medical practice is input in addition to the number of elapsed days and the medical information, instead of performing the weighting on the prediction probability output by the prediction model 24 or the number of elapsed days input to the prediction model 24.

In the above-described embodiment, the fall prediction information is derived only in a case in which the number of elapsed days is equal to or smaller than the threshold value Th1, but the present disclosure is not limited to this. The fall prediction information may be derived for all elapsed days.

In addition, in the above-described embodiment, the scheduled hospital visit date is used as the prediction date information, but the present disclosure is not limited to this. Information on any date designated by an operator can be used as the prediction date information.

In addition, in the above-described embodiment, the contribution degree is derived, but the present disclosure is not limited to this. The derivation of the contribution degree and the notification may be omitted.

In addition, in the above-described embodiment, all of the basic patient information, the disease information, the drug information, the surgery information, the examination information, and the keyword information are input to the prediction model 24 as the medical information to derive the fall prediction information, but the present disclosure is not limited to this. Only a part of the basic patient information, the disease information, the drug information, the surgery information, the examination information, and the keyword information may be input to the prediction model 24 to derive the fall prediction information.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the medical support program 12 is stored (installed) in advance in the storage unit 13, but this is not limited to this. The medical support program 12 may be provided in a form recorded on a recording medium such as a CD-ROM (Compact Disc Read Only Memory), DVD-ROM (Digital Versatile Disc Read Only Memory), and USB (Universal Serial Bus) memory. In addition, the medical support program 12 may be provided in a form that the medical support program 12 is downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs.

Appendices of the present disclosure will be described below.

### Appendix 1

A medical support device comprising:
a processor,
in which the processor
   acquires medical information including date information associated with a medical practice for a target patient and prediction date information for predicting a fall of the target patient,
   derives fall prediction information of the target patient using a prediction model that has been trained through machine learning to predict the fall of the target patient based on the number of elapsed days derived from the date information and the prediction date information, and
   notifies of the fall prediction information.

### Appendix 2

The medical support device according to Appendix 1,
in which the prediction date information is a scheduled hospital visit date of the target patient.

### Appendix 3

The medical support device according to Appendix 1,
in which the processor
   acquires the prediction date information, and
   sets, as the target patient, a patient for whom the prediction date information is associated as a scheduled hospital visit date.

### Appendix 4

The medical support device according to Appendix 1 or 2,
in which the date information is at least one of a prescription date of a drug for the target patient, an examination date for the target patient, a surgery date for the target patient, a hospitalization date of the target patient, a discharge date of the target patient, or a diagnosis date for the target patient.

### Appendix 5

The medical support device according to any one of Appendices 1 to 4,
in which, in a case in which a plurality of pieces of the date information are associated with the medical practice for the target patient in the medical information, the processor
   acquires specific date information closest to the prediction date information among the plurality of pieces of date information, and
   derives the fall prediction information of the target patient using the prediction model based on the number of elapsed days derived from the specific date information and the prediction date information.

### Appendix 6

The medical support device according to Appendix 5,
in which the processor derives the fall prediction information weighted based on the number of consecutive days on which the medical practice is performed from the specific date information.

### Appendix 7

The medical support device according to Appendix 5,
in which the processor derives the fall prediction information weighted based on the number of days on which the medical practice is performed within a predetermined period from the specific date information or the prediction date information.

### Appendix 8

The medical support device according to any one of Appendices 1 to 4,
in which the processor compares the number of elapsed days with a predetermined first threshold value, and derives the fall prediction information based on the number of elapsed days that is equal to or smaller than the first threshold value.

### Appendix 9

The medical support device according to Appendix 1,
in which the first threshold value is set to a different value for each classification of the medical information associated with the date information.

### Appendix 10

The medical support device according to any one of Appendices 1 to 5,
in which the medical information includes at least one of basic patient information, disease information, drug information, surgery information, examination information, or keyword information,
the date information is included in at least one of the basic patient information, the disease information, the drug information, the examination information, or the surgery information, and
the processor derives the fall prediction information using the prediction model based on the number of elapsed days and at least one of the basic patient information, the disease information, the drug information, the surgery information, the examination information, or the keyword information.

### Appendix 11

The medical support device according to Appendix 10,
in which the processor
   derives a contribution degree of the medical information and the number of elapsed days input to the prediction model to the fall prediction information, and
   notifies of at least one of the medical information or the number of elapsed days based on the contribution degree together with the fall prediction information.

### Appendix 12

The medical support device according to any one of Appendices 1 to 11,
in which the fall prediction information is at least one of presence or absence of a fall risk or a probability of the fall risk,
the fall prediction information is used for determining whether or not the fall risk is present or the fall risk is equal to or greater than a second threshold value, and
the processor notifies of the fall prediction information based on a result of the determination.

### Appendix 13

The medical support device according to Appendix 12,
in which the processor notifies a medical worker associated with the target patient of the fall prediction information.

### Appendix 14

A medical support method comprising:
acquiring medical information including date information associated with a medical practice for a target patient and prediction date information for predicting a fall of the target patient;
deriving fall prediction information of the target patient using a prediction model that has been trained through machine learning to predict the fall of the target patient based on the number of elapsed days derived from the date information and the prediction date information; and
notifying of the fall prediction information.

### Appendix 15

A medical support program causing a computer to execute:
a procedure of acquiring medical information including date information associated with a medical practice for a target patient and prediction date information for predicting a fall of the target patient;
a procedure of deriving fall prediction information of the target patient using a prediction model that has been trained through machine learning to predict the fall of the target patient based on the number of elapsed days derived from the date information and the prediction date information; and
a procedure of notifying of the fall prediction information.

### Explanation of References

What is claimed is:

1. A medical support device comprising:
a processor,
wherein the processor
acquires medical information including date information associated with a medical practice for a target patient and prediction date information for predicting a fall of the target patient,
derives fall prediction information of the target patient using a prediction model that has been trained through machine learning to predict the fall of the target patient based on the number of elapsed days derived from the date information and the prediction date information, and
notifies of the fall prediction information.

2. The medical support device according to claim 1,
wherein the prediction date information is a scheduled hospital visit date of the target patient.

3. The medical support device according to claim 1,
wherein the processor
acquires the prediction date information, and
sets, as the target patient, a patient for whom the prediction date information is associated as a scheduled hospital visit date.

4. The medical support device according to claim 1,
wherein the date information is at least one of a prescription date of a drug for the target patient, an examination date for the target patient, a surgery date for the target patient, a hospitalization date of the target patient, a discharge date of the target patient, or a diagnosis date for the target patient.

5. The medical support device according to claim 1,
wherein, in a case in which a plurality of pieces of the date information are associated with the medical practice for the target patient in the medical information, the processor
acquires specific date information closest to the prediction date information among the plurality of pieces of date information, and
derives the fall prediction information of the target patient using the prediction model based on the number of elapsed days derived from the specific date information and the prediction date information.

6. The medical support device according to claim 5,
wherein the processor derives the fall prediction information weighted based on the number of consecutive days on which the medical practice is performed from the specific date information.

7. The medical support device according to claim 5,
wherein the processor derives the fall prediction information weighted based on the number of days on which the medical practice is performed within a predetermined period from the specific date information or the prediction date information.

8. The medical support device according to claim 1,
wherein the processor compares the number of elapsed days with a predetermined first threshold value, and derives the fall prediction information based on the number of elapsed days that is equal to or smaller than the first threshold value.

9. The medical support device according to claim 8,
wherein the first threshold value is set to a different value for each classification of the medical information associated with the date information.

10. The medical support device according to claim 1,
wherein the medical information includes at least one of basic patient information, disease information, drug information, surgery information, examination information, or keyword information,
the date information is included in at least one of the basic patient information, the disease information, the drug information, the examination information, or the surgery information, and
the processor derives the fall prediction information using the prediction model based on the number of elapsed days and at least one of the basic patient information, the disease information, the drug information, the surgery information, the examination information, or the keyword information.

11. The medical support device according to claim 10,
wherein the processor
derives a contribution degree of the medical information and the number of elapsed days input to the prediction model to the fall prediction information, and
notifies of at least one of the medical information or the number of elapsed days based on the contribution degree together with the fall prediction information.

12. The medical support device according to claim 1,
wherein the fall prediction information is at least one of presence or absence of a fall risk or a probability of the fall risk,
the fall prediction information is used for determining whether or not the fall risk is present or the fall risk is equal to or greater than a second threshold value, and
the processor notifies of the fall prediction information based on a result of the determination.

13. The medical support device according to claim 12,
wherein the processor notifies a medical worker associated with the target patient of the fall prediction information.

14. A medical support method comprising:
acquiring medical information including date information associated with a medical practice for a target patient and prediction date information for predicting a fall of the target patient;
deriving fall prediction information of the target patient using a prediction model that has been trained through machine learning to predict the fall of the target patient based on the number of elapsed days derived from the date information and the prediction date information; and
notifying of the fall prediction information.

15. A non-transitory computer-readable storage medium that stores a medical support program causing a computer to execute:
a procedure of acquiring medical information including date information associated with a medical practice for a target patient and prediction date information for predicting a fall of the target patient;
a procedure of deriving fall prediction information of the target patient using a prediction model that has been trained through machine learning to predict the fall of the target patient based on the number of elapsed days derived from the date information and the prediction date information; and
a procedure of notifying of the fall prediction information.
